(19) United States
(12) Patent Application Publication
(10) Pub. No.: US 2022/0313935 A1
(43) Pub. Date: Oct. 6, 2022

(54) Medical Apparatus

(71) Applicant: Scott Airway Management Pty Ltd., Red Hill (AU)

(72) Inventor: Paul Scott, Red Hill (AU)

(21) Appl. No.: 17/617,047

(22) PCT Filed: Jun. 4, 2020
(86) PCT No.: PCT/AU2020/050568

§ 371 Date: Dec. 7, 2021

(30) Foreign Application Priority Data
Jun. 7, 2019 (AU) 2019901980

(51) Int. Cl. A61M 16/06; A61M 16/04

(52) U.S. Cl. CPC A61M 16/0616; A61M 16/0493; A61M 16/047; A61M 2205/0216

(57) ABSTRACT

A sealing apparatus for use in combination with an oropharyngeal device for maintaining a patient airway, the oropharyngeal device having a hollow airway tube with proximal end and a distal end, the proximal end being adapted to be fluidly coupled with a supply of gas for passage of air or medical gas wherein the device is adapted for insertion through the mouth of a patient and with one end into the pharynx of a patient above and spaced from the epiglottis of the patient, the sealing apparatus comprising: a flexible sheet adapted to be placed over a user's face; and the flexible sheet comprising an opening to allow the distal end of the tube to be passed therethrough, the opening being dimensioned to prevent the proximal end of the tube from being passed through the opening.

### CROSS-REFERENCE TO RELATED APPLICATIONS

This application is the national stage application of International Application No. PCT/AU2020/050568, filed Jun. 4, 2020, which designates the United States of America. This application also claims priority, under 35 U.S.C. § 119, to Australian Patent Application No. 2019901980, filed Jun. 7, 2019. The prior applications are herein incorporated by reference in their entirety.

### TECHNICAL FIELD

The present invention relates to medical apparatus that may be useful for use in combination with an oropharyngeal device for establishing and maintaining an airway.

### BACKGROUND

Any references to methods, apparatus or documents of the prior art are not to be taken as constituting any evidence or admission that they formed, or form part of the common general knowledge.

The oropharyngeal airway tube (also known as a Guedel airway) is one way of achieving and maintaining an airway between the patient's mouth and larynx. The conventional Guedel airway bypasses the oropharynx and allows air (oxygen, sedating gas, etc.) to bypass the tongue and upper pharynx. The Guedel airway is usually used for patients who may be incompletely conscious, as its insertion into the face of an alert person may not be well received particularly if the user is conscious.

FIG. 1 shows a currently known Guedel airway 100 which is a curved device to be inserted into the mouth and designed to maintain the airway between the patient's mouth and larynx whilst also prevent the patient biting by providing a bite resistant tube 105 that can be placed in the patient's mouth. The curved tube 105 rests between the posterior pharyngeal wall and the tongue, keeping the epiglottis off the posterior pharyngeal wall (where it would normally be forced into by the flaccid collapse of the tongue).

Maintenance of a patient's airway is of paramount importance at the time of administering anaesthesia. Difficulties or failures in managing the airway are the major factors underlying morbidity and mortality relating to anaesthesia. With the loss of consciousness caused by general anaesthesia, there is loss of protective airway reflexes (such as coughing), loss of airway patency and sometimes loss of a regular breathing pattern due to the effect of anaesthetics, opioids, or muscle relaxants. To maintain an open airway and regulate breathing within acceptable parameters, some form of an orthopharyngeal airway tube is usually inserted in the airway after the patient is unconscious. At all times during the course of anaesthesia and resuscitation, the anaesthetist needs to maintain a patent airway and be able to seal the breathing circuit for positive pressure ventilation. Positive pressure ventilation can be delivered either manually using a bag and valve technique or by using a mechanical ventilator.

When using an anaesthetic face mask, the anaesthetist has to maintain a patient airway and at the same time seal the mask around the patient's mouth and nose while using an oropharyngeal airway (OPA). It has been found that it is extremely difficult for an anaesthetist to maintain a seal between the mask and the patient's face when patients have a beard or a moustache or have facial abnormalities (such as heavy scarring or sunken cheeks etc.). It has also been found that during the time of inserting the Guedel airway into the patient's mouth there remains a possibility of the patient coughing (in reaction to the insertion of the Guedel airway) which might lead to airborne droplets being released from the patient's mouth. When patients are not able to breathe for themselves, healthcare workers cover the patient's face with a mask to force air into the patient's lungs to keep them alive. This is called Bag-Mask Ventilation (BMV). The success of this action relies on the ability to form a perfect seal between the mask and the patient's face, delivering oxygen at a pressure high enough to overcome the resistance of the airways, lungs and chest wall. If there is an inadequate seal between the face and mask, there is potential aerosolisation of airway secretions. Aerosolised airway secretions can contain viral particles which can then lead to viral transmission.

It is also important to realise that there can be other scenarios where medical apparatus is used to maintain an airway in a patient. For example, a patient airway needs to be maintained when instruments, such as endoscopes, are inserted through a person's mouth into the human body, in medical procedures. To prevent the patient from biting into the instrument, a bite block is used which is generally a plastic tube which is positioned in the patient's open mouth. The bite block can often be provided with oxygen supply ports which assist the patient in breathing and maintaining the airway whilst an endoscopic procedure is being carried out. The instrument is then inserted through the bite block opening into the stomach or other areas while the patient continues to breathe.

In healthcare institutions, and public health settings, however, it is desirable not only to protect the patient from potentially harmful particulate matter, including biological pathogens but also to protect populations from a patient exhaling such pathogens into the environment where other healthcare providers may be present. It is therefore desirable to provide medical apparatus that can effectively protect the patient and surrounding healthcare providers from contacting any airborne viruses or bacterial that may otherwise spread during procedures such as endoscopy or providing breathing assistance due to involuntary coughing by the patient whilst they are being attended to by healthcare providers.

### SUMMARY OF INVENTION

In an aspect, the invention provides a sealing apparatus for use in combination with an oropharyngeal device for maintaining a patient airway, the oropharyngeal device having a hollow airway tube with proximal end and a distal end, the proximal end being adapted to be fluidly coupled with a supply of gas for passage of air or medical gas wherein the oropharyngeal device is adapted for insertion through the mouth of a patient and with one end into the pharynx of a patient above and spaced from the epiglottis of the patient, the sealing apparatus comprising:
i. a flexible sheet adapted to be placed over a user's face; and
ii. the flexible sheet comprising an opening to allow the distal end of the tube to be passed therethrough, the opening being dimensioned to prevent the proximal end of the tube from being passed through the opening.

In an embodiment, the sheet is substantially impervious to air or one or more of the other medical gases.

In an embodiment, the proximal end of the device comprises a circumferential flange and wherein width of the opening of the sealing apparatus is dimensioned to be smaller than a width/diameter of the circumferential flange to prevent the proximal end of the device from passing through the opening of the flexible sheet.

In an embodiment, the opening of the flexible sheet is dimensioned such that at least a portion of an underside surface of the circumferential flange contacts a portion of the flexible sheet defining the opening to thereby form a seal.

In an embodiment, the flexible sheet extends between two lateral sides with top and bottom portions of the sheet extending therebetween and wherein the top portion comprises a cut-out section for receiving a nose of the user, the cut-out section being flanked by a top left portion and a top right portion of the flexible sheet.

In an embodiment, the opening of the sealing apparatus is located below the cut-out section for being positioned substantially adjacent the mouth of the user.

In an embodiment, the opening of the sealing apparatus is shaped to substantially correspond to a profile or cross section of the tube of the device.

In an embodiment, the sealing apparatus is formed from medical grade polymeric or silicone material.

In an embodiment, the sealing apparatus further comprises one or more tabs to facilitate attachment of the sealing apparatus to the user's face during use.

In another aspect, the invention also provides a kit comprising the sealing apparatus as described in the previous aspect in combination with an oropharyngeal device having a hollow airway tube with proximal end and a distal end, the proximal end being adapted to be fluidly coupled with a supply of gas for passage of air or medical gas wherein the device is adapted for insertion through the mouth of a patient and with one end into the pharynx of a patient above and spaced from the epiglottis of the patient.

In yet another aspect, the invention provides an oropharyngeal device having a hollow airway tube with proximal end and a distal end, the proximal end being adapted to be fluidly coupled with a supply of gas for passage of air or medical gas wherein the device is adapted for insertion through the mouth of a patient and with one end into the pharynx of a patient above and spaced from the epiglottis of the patient; and a seal comprising: a flexible sheet adapted to be placed over a user's face wherein the flexible sheet comprises an opening to allow the distal end of the tube to be passed therethrough, the opening being dimensioned to prevent the proximal end of the tube from being passed through the opening.

In an embodiment, outer walls of the tube comprise a plurality of projecting members located adjacent the proximal end of the tube to facilitate positioning and/or retention of the sealing apparatus below the circumferential flange of the device.

In yet another aspect, the invention provides a sealing assembly adapted for contacting a user's face, the sealing assembly comprising a flexible sheet adapted to be placed over the user's face, the sheet being substantially impervious to air or gases, wherein the flexible sheet defines an opening to allow access into the user's mouth and wherein the flexible sheet is dimensioned for receiving and contacting an anesthetic face mask to form a seal between the facemask and at least a portion of the sheet during use.

In another aspect, the invention provides a medical apparatus for use in combination with an airway device to maintain a patient airway, the airway device comprising a proximal end and a distal end with a passage extending therebetween wherein the proximal end is adapted to receive a supply of gas for passage of air or medical gas and wherein the distal end is adapted to be inserted through the mouth of the patient, the medical apparatus comprising: a flexible sheet adapted to be placed over a user's face wherein the flexible sheet comprises an opening to allow the distal end of the tube to be passed therethrough, the opening being dimensioned to prevent the proximal end of the tube from being passed through the opening.

In an embodiment, the sheet is substantially impervious to air or liquids or one or more of the other medical gases.

In an embodiment, the proximal end or the distal end of the airway device comprises a circumferential flange and wherein width of the opening of the in the sheet is dimensioned to be smaller than the width/diameter of the circumferential flange to prevent the proximal end or the distal end from passing through the opening of the mask and becoming uncoupled from the airway device.

In an embodiment, the opening of the flexible sheet is dimensioned such that at least a portion of an underside surface of the circumferential flange contacts a portion of the flexible sheet defining the opening to form a seal.

In an embodiment, the flexible sheet extends between two lateral sides with top and bottom portions of the sheet extending therebetween and wherein the top portion comprises a cut-out section for receiving a nose of the user, the cut-out section being flanked by a top left portion and a top right portion of the flexible sheet.

In an embodiment, the opening is located below the cut-out section for being positioned substantially above the mouth of the user.

In an embodiment, the medical apparatus further comprises a locking member configured to be received and engaged with a locking portion located on an outer wall portion of the passage extending between the proximal end and distal end of the airway device for detachably coupling said medical apparatus with the airway device.

In an embodiment, the locking member comprises a profiled projection member configured for engagement with an opening in the locking portion.

In an embodiment, during use the projection member extends in a transverse direction relative to a longitudinal axis of the passage.

In an embodiment, the projection member extends in an in-use downwardly direction towards a radially central portion of the passage.

In an embodiment, the medical apparatus further comprises a passage engaging wall portion formed integrally with said flexible sheet, the wall portion being dimensioned for surrounding and engaging an outer wall of the passage, the passage engaging wall defining the opening to allow the distal end of the tube to pass through at an end portion of the passage engaging wall.

In an embodiment, at least a portion of the flexible sheet, during use, engages with a back wall of the front piece for the bite block.

In an embodiment, said at least portion of the flexible sheet is integrally formed with the passage engaging wall portion.

In an embodiment, the flexible sheet is configured to be spaced away from hook members projecting from the front piece of the bite block, during use.

In another aspect, the invention provides a mask for use in combination with an endoscopy bite block including a front piece to fit over a patient's mouth which has a central opening communicating with a passage extending into the patient's mouth to receive and pass an endoscopic tube, the mask comprising: a flexible sheet adapted to be placed over a user's face; wherein the flexible sheet comprises opening to allow the passage of the bite block to be passed therethrough, the opening being dimensioned to prevent the front piece from being passed through the opening.

In an embodiment, the sheet is substantially impervious to air or liquid.

In an embodiment, the passage extends between a proximal end located adjacent the central opening on the front piece and a distal end adapted to be inserted into the mouth of the patient and being located away from the front piece, wherein the distal end comprises a circumferential flange and wherein width of the opening of the mask is dimensioned to be smaller than the width or diameter of the circumferential flange to prevent the distal end of the passage from passing through the opening of the mask and becoming uncoupled from the endoscopy bite block.

In an embodiment, the flexible sheet extends between two lateral sides with top and bottom portions of the sheet extending therebetween and wherein the top portion comprises a cut-out section for receiving a nose of the user, the cut-out section being flanked by a top left portion and a top right portion of the flexible sheet.

In an embodiment, the opening of the mask is located below the cut-out section for being positioned substantially above the mouth of the user.

In an embodiment, the mask comprises a locking member configured to be received and engaged with a locking portion located on an outer wall portion of the passage for detachably coupling said mask with the bite block.

In an embodiment, the locking member comprises a profiled and flexible projection member configured for engagement with an opening in the locking portion.

In an embodiment, during use, the projection member extends in a transverse direction relative to a longitudinal axis of the passage.

In an embodiment, the projection member extends in an in-use downwardly direction towards a radially central portion of the passage.

In an embodiment, the mask comprises a passage engaging wall portion formed integrally with said flexible sheet, the wall being dimensioned for surrounding and engaging an outer wall of the passage, the passage engaging wall defining the opening at an end portion of the passage engaging wall.

In an embodiment, a portion of the flexible sheet, during use, engages with a back wall of the front piece for the bite block.

In an embodiment, said at least portion of the flexible sheet is integrally formed with the passage engaging wall portion.

In an embodiment, the flexible sheet is configured to be spaced away from hook members projecting from the front piece, during use.

In another aspect, the invention provides an endoscopy bite block in combination with the mask as described herein.

In yet another aspect, the invention provides an endoscopy bite block in combination with a mask, wherein the bite block comprises a front piece for fitting over a patient's mouth, the front piece having a central opening communicating with a passage extending into the patient's mouth to receive and pass an endoscopic tube therethrough and wherein the mask comprises a flexible sheet adapted to be placed over a user's face; wherein the flexible sheet comprises opening to allow the passage to be passed therethrough, the opening being dimensioned to prevent the front piece from being passed through the opening.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is a side view of an oropharyngeal tube 100 (also known as a Guedel oral airway) as known in the prior art.

FIG. 2 is frontal view of a sealing apparatus 200 in accordance with a first embodiment of the present invention.

FIG. 3 is an isometric view of the sealing apparatus 200.

FIG. 4 is a side view of the sealing apparatus 200.

FIG. 5A is an isometric view of the sealing apparatus 200 wherein the oropharyngeal tube 100 is being inserted into the opening 210 of the sealing apparatus 200.

FIG. 5B shows the sealing apparatus 200 in a coupled configuration with the oropharyngeal tube 100.

FIG. 5C shows the sealing apparatus 200 in a coupled configuration with the oropharyngeal tube 100 in an in-use configuration.

FIG. 6 is a rear sectional perspective view (exploded) of a mask 500 and an endoscopic bite block 600 in accordance with a second embodiment.

FIG. 7 is a rear perspective view of the mask 500 shown in a coupled configuration with the endoscopic bite block 600.

FIG. 8 is a side and frontal perspective view of the mask 500 shown in a coupled configuration with the endoscopic bite block 600.

FIG. 9 is an enlarged side and rear view of the mask 500 shown in a coupled configuration with the endoscopic bite block 600.

FIG. 10 is a frontal view of the mask 500 shown in a coupled configuration with the endoscopic bite block 600.

FIG. 11 is an isolated sectional view of the mask 500 across section A-A′.

FIG. 12 is an isolated sectional view of the mask 500 across section B-B′.

### DETAILED DESCRIPTION OF PREFERRED EMBODIMENTS

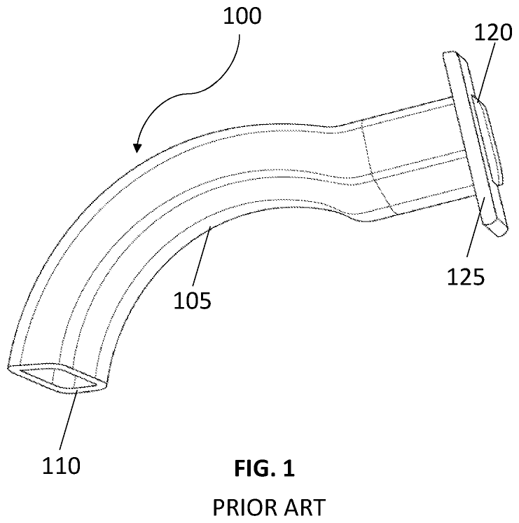

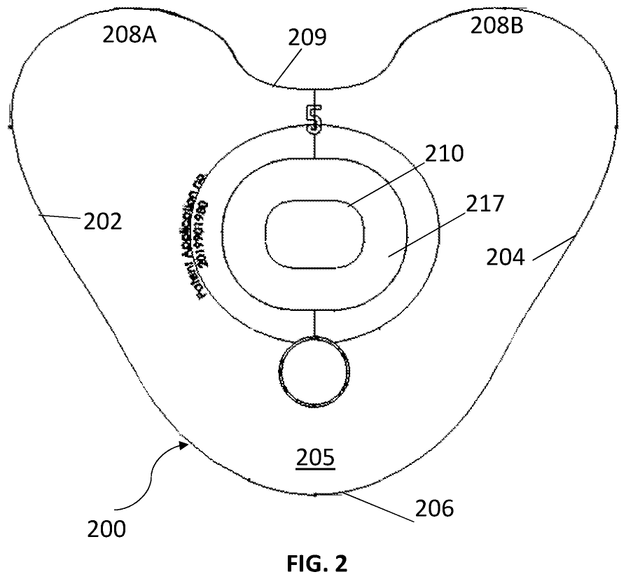

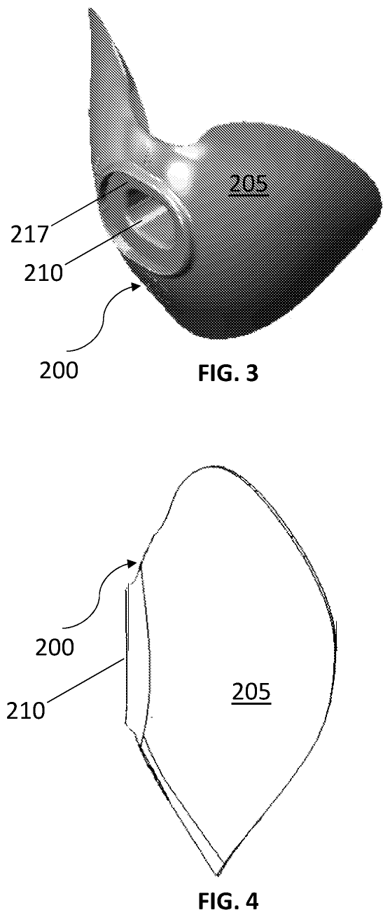

FIG. 1 illustrates an oropharyngeal device 100 as known in the prior art. The device 100 comprises a curved tube 105 that is typically formed from a biologically inert polymer. The curved tube 105 offers a bite resistant passage for air or medical gases from the user's lips to the user's posterior pharynx. The curved tube 105 extends between a proximal end 120 and a distal end 110. The proximal end 120, during use, is typically positioned close to the outside of the user's mouth whilst the distal end 110 is inserted through the mouth. The curved tube 105 is made from rigid material with a kink resistant curve to push the tongue forward keeping the epiglottis off the posterior pharyngeal wall. The distal end 110 is provided with tapered edges to reduce oropharyngeal mucosal trauma during insertion of the device 100 into the user's mouth. The proximal end 120 is provided with a circumferential flange 125 that limits the depth of insertion of the device 100 and prevents the device 100 from being accidentally lost inside the patient's airway.

In a preferred embodiment, the invention provides a sealing apparatus 200 that is suitable for use in combination with the oropharyngeal device 100. The sealing apparatus 200 comprises a flexible sheet 205 that is substantially impervious to air or any other gas and is suitable for being placed over the user's face. The sealing apparatus 200 also comprises an opening 210 that allows the curved tube 105 to be passed through the opening 210. Specifically, during use, the distal end 110 of the oropharyngeal device 100 is inserted into the opening 210 and the curved and rigid tube 105 is passed through the opening of the sealing apparatus 200. The opening 210 is sized or dimensioned such that the circumferential flange 225 is not able to accidentally pass through the opening 210 of the sealing apparatus 200. The flexible sheet 210 surrounding and defining the opening 210 contacts an underside portion 224 of the circumferential flange 225 and assists with achieving a good seal around the proximal end opening 220 particularly on users with facial hair.

During use, as the tube 105 is inserted into the patient's mouth, the flexible sheet 205 of the sealing apparatus 200 is positioned over the user's face (which may have facial hair or oily skin). Advantageously, the sealing apparatus 200 is specifically shaped to allow the sealing apparatus 200 to be easily positioned over the user's face. It has been found by the inventor that the use of the sealing apparatus 200 provides a significant improvement in achieving a seal during bag mask ventilation of patients with beards or facial hair. The placement of the flexible sheet of the sealing apparatus 200 around the circumferential flange 125 during bag mask ventilation allows the bag mask to form a seal and prevents air or any other medical gases from escaping. The region surrounding the opening 210 comprises a complementary configuration in the form of a recessed flange 217 to receive and engage the circumferential flange 125. The use of the oropharyngeal device 100 without the sealing apparatus 200 on bearded patients may result in air or medical gases escaping between the face and the mask through the beard. Such a lack of sealing makes it very difficult to achieve an adequate seal to allow ventilation which can lead to lack of oxygenation eventually leading to deterioration of patient health. The use of the sealing apparatus 200 addresses this problem and helps in achieving an improved seal between the mask and the patient's face and addresses a very important problem in the art.

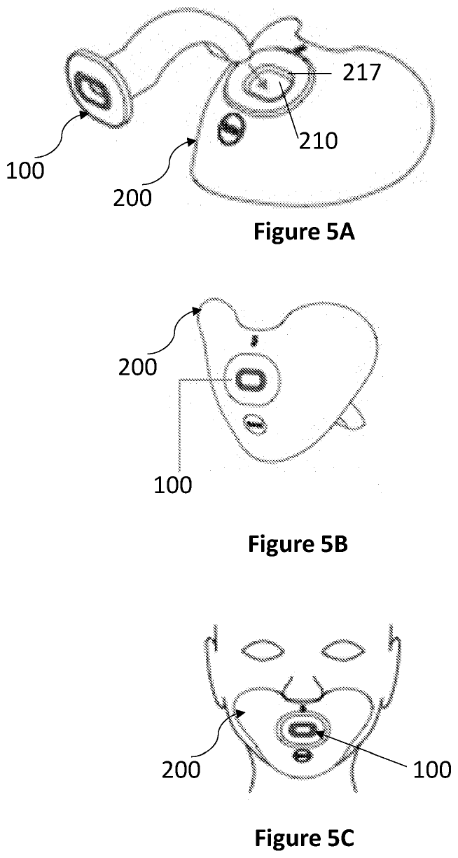

In the coupled position, shown best in FIGS. 5B and 5C, the opening 210, especially the flanged recess 217 of the flexible sheet 205 is positioned around the circumferential flange 125 of the airway device 100 and the flexible sheet 205 extends over the patient's face to surround the mouth region of the patient. As a result, any droplets inadvertently released by the patient (whilst the oropharyngeal device 100 is in the patient's mouth) is captured along the inner walls of the flexible panel 205 positioned in close proximity to the user's mouth. As a result, the sealing apparatus 200 additionally prevents the spread of such droplets and makes the working environment for the healthcare providers much safer and more hygienic. By providing a flexible and contoured surface along the inner walls of the flexible sheet 205, the sealing apparatus 200 creates a seal with the mask and an open airway for air to flow down to the lungs of the patient. Any aerosols that are generated while the patient is intubated are directed and captured on the inner walls or posterior surface of the flexible sheet 205 to form a physical barrier or shield between the patient secretions and the healthcare workers.

The sealing apparatus 200 in the preferred embodiment extends between lateral sides 202 and 204 with a lower portion 206 and an upper portion (generally denoted by 208). The upper portion 208 includes a cut-out section 209 to accommodate space for the user's nose. The cut-out section 209 is flanked by top portions 208A and 208B that may be positioned over the cheeks of the user. The sealing apparatus 200 may also be provided with attachment tabs for securing the sealing apparatus to the user's face. The attachment tabs may be provided with an adhesive backing to facilitate the securement of the sealing apparatus 200 to the user's face. The location of the tabs may be varied to suit users with varying facial sizes. The overall size or dimensions of the sealing apparatus 200 may also be varied to suit users of various sizes and facial structures. Finally, the size of the opening 210 in the sealing apparatus 200 may also be varied in conjunction with the size of the oropharyngeal device 100. It is desirable to provide a sealing apparatus 200 in which the size of the opening 210 corresponds substantially to the size of the tube 105 of the oropharyngeal device 100.

In at least some embodiments, the invention is directed to not only the sealing apparatus 200 but also a kit comprising the sealing apparatus 200 in combination with the oropharyngeal device 100.

In some further alternative embodiments, the sealing apparatus 200 may also be formed in combination with the oropharyngeal device 100 as a single device. In such an embodiment, the portion of the flexible sheet that defines the opening 210 may be fastened to an underside portion of the flange 125 of the oropharyngeal device 100 to provide the desired sealing effect.

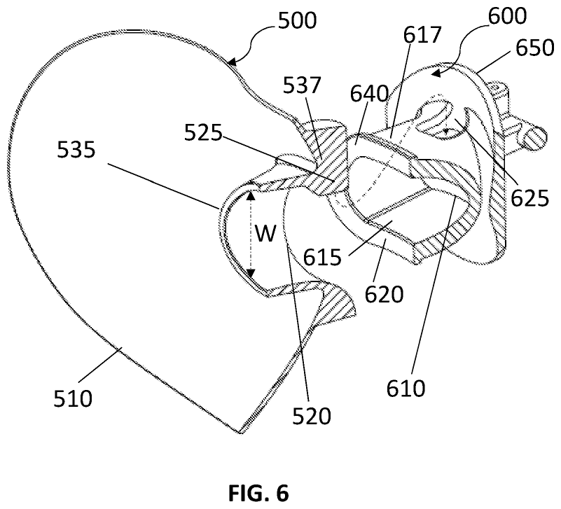

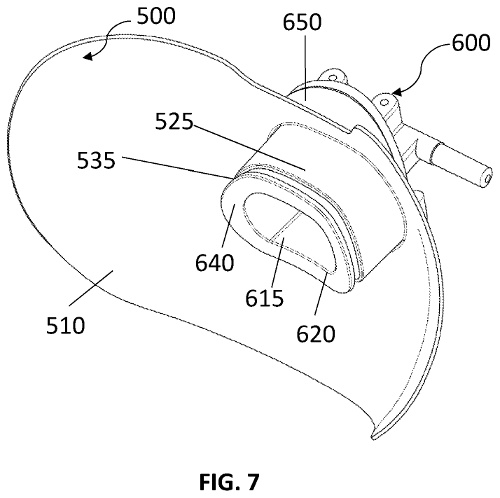

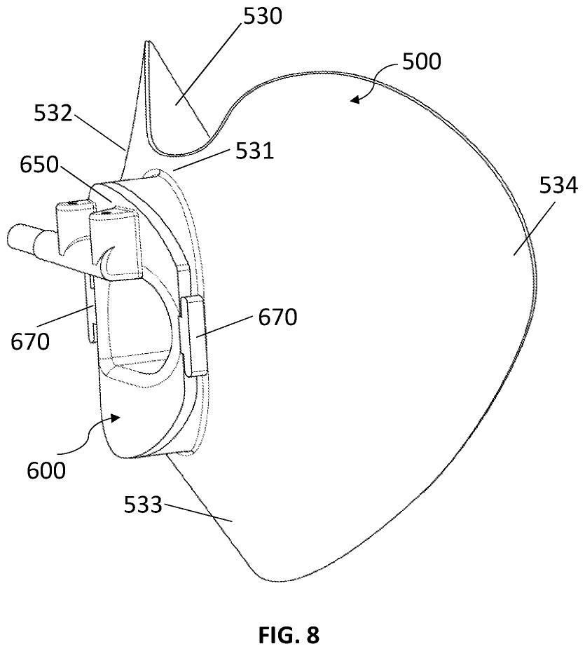

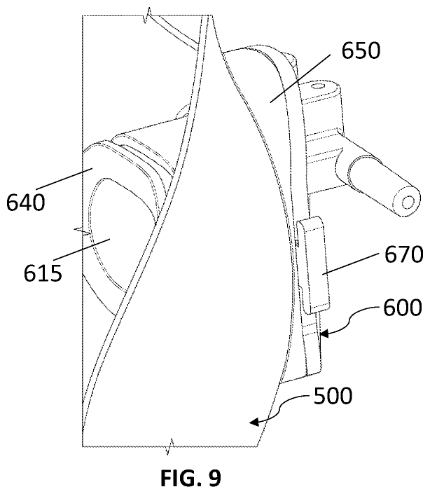

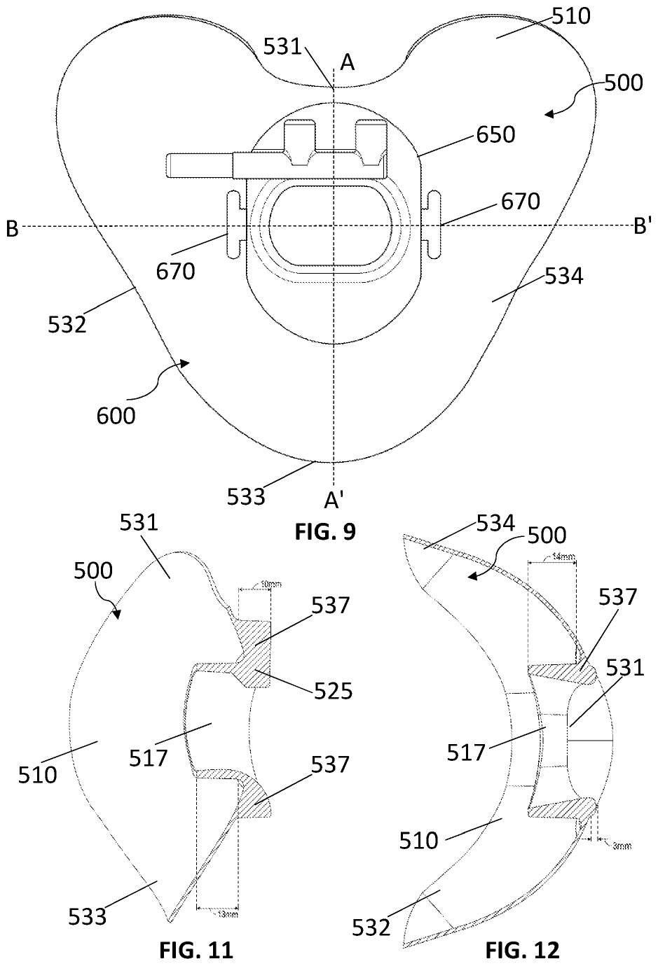

Referring to FIGS. 6 to 12, a second embodiment of the medical apparatus in the form of a mask 500 for use in combination with an endoscopic bite block 600 has been illustrated. The mask 500 is provided to shield healthcare providers from being accidentally exposed to infectious airborne droplets released by a patient while they have the endoscopic bite block 600 in their mouth. The bite block 600 shown in FIGS. 6 to 12 including a front piece 650 to fit over a patient's mouth which has a central opening communicating with a passage 615 extending into the patient's mouth to receive and pass an endoscopic tube. The passage 615 that extends between the proximal end 610 (that receives the endoscope tube) and a distal end 620 (which is generally located inside the patient's mouth while endoscopic examination is underway. The passage 615 is sufficiently large to accommodate an endoscopy tube (not shown) to be passed through and allow some residual volume around the endoscopy tube which allows air to pass through the passage 615 so that the patient can utilize that air for breathing while endoscopic examination is being carried out.

The mask 500 is configured to be detachably coupled with the bite block 600 (as will be explained in further detail) comprises a flexible sheet 510 that can be placed over the patient's face area surrounding the mouth of the patient. The flexible sheet 510 is sufficiently flexible to confirm to the contours of the patient's face. The flexible sheet 510 comprises an opening 520 that is located radially inwardly relative to the peripheral portions of the flexible sheet 510. The opening 520 is sized to allow the distal end 620 of the bite block 600 to pass through the opening 520. It is important to note that the opening 520 is dimensioned to be smaller than the width/diameter (denoted by W in FIG. 6) of a circumferential flange 640 located at the distal end 620 to prevent the distal end 620 from withdrawing through the opening 520 of the mask 500 and becoming uncoupled from the bit block 600.

The sheet 510 extends between two sides 532 and 534 with top and bottom portions 531 and 533 of the sheet 510 extending therebetween with the opening 520 being located substantially centrally relative to the peripheral portions of the sheet 510. The top portion 531 comprises a cut-out section 530 for receiving a nose of the user, the cut-out section 530 being flanked by a top left portion and a top right portion of the flexible sheet 510 to allow the cut-out section 530 to be positioned substantially above the mouth of the user when the mask 500 is coupled with the bite block 600 during use.

The mask 500 includes a passage engaging portion 535 that is formed integrally with the flexible sheet 510 that is dimensioned to surround and engage an outer wall 617 of the passage 615. The passage engaging portion 535 extends from the opening 520 (that is located adjacent the proximal end 610) towards the distal end 620 of the passage 615. The passage engaging portion 535 comprises the wall(s) that defines the opening 520 in the mask 500 within which the passage 615 of the bite block 600 is inserted. The passage engaging portion 535 of the mask 500 is also flexible and is dimensioned to snap onto the outer walls of the passage 615 which prevents the mask 500 from becoming decoupled from the bite block 600. Advantageously, thickness of the passage engaging portion 535 is slightly greater than thickness of the flexible sheet 510 (best shown in FIGS. 11 and 12). The passage engaging portion 535 is formed continuously with a thickened turned portion 537 that is connected with the flexible sheet 510. A part of the flexible sheet portion 510 contacts a back face of the front piece 650 of the bite block 600 during use in the coupled configuration.

The mask 500 also includes a profiled and flexible locking member 525 that is configured to be received and engaged with a locking portion 625 located on the outer wall portion 617 of the passage 615 for detachably coupling said mask with the bite block. In the preferred embodiment, the locking member 525 extends downwardly in a transverse direction relative to a longitudinal axis of the passage provided in the bite block 600. The locking arrangement provided by the locking member 525 prevents the mask 500 from becoming decoupled from the bite block 500.

The front piece 650 for the bite block 600 also includes projecting holding members 670 that are maintained in a spaced relationship relative to an outer walls of the flexible sheet 510 to allow the user to hold the holding members 670 and pull the mask 500 away from the coupled bite block 600 to release the locking member 525 out of the locking portion 625.

In the coupled position, the flexible sheet 510 surrounds the front piece 650 and extends over the patient's face to surround the mouth region of the patient. As a result, any droplets inadvertently released by the patient (whilst endoscopy is being conducted) is captured along the inner walls of the flexible sheet 510 positioned in close proximity to the user's mouth. As a result, the mask 500 prevents the spread of such droplets and makes the working environment for the healthcare providers much safer and hygienic.

In some embodiments, the mask 500 may be integrally formed with the bite block 600. In such an embodiment, the flexible sheet 510 may be integrally with the front piece 650 of the bite block 600 and the locking arrangement as previously described may not be necessary. It would also be understood that one or ports may be provided in the bite block for additionally supplying oxygen or other gases into the mouth of the patient.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

1. Medical Apparatus for use in combination with an airway device to maintain a patient airway, the airway device comprising a proximal end and a distal end with a passage extending therebetween wherein the proximal end is adapted to receive a supply of gas for passage of air or medical gas and wherein the distal end is adapted to be inserted through the mouth of the patient, the medical apparatus comprising:
a flexible sheet adapted to be placed over a user's face wherein the flexible sheet comprises an opening to allow the distal end of the tube to be passed therethrough, the opening being dimensioned to prevent the proximal end of the tube from being passed through the opening, and wherein the flexible sheet is substantially impervious to air or liquids or one or more of the other medical gases; and,
a passage engaging wall formed integrally with said flexible sheet, the passage engaging wall being dimensioned for surrounding and engaging an outer wall of the passage of the mouthpiece.

2. (canceled)

3. Medical apparatus in accordance with claim 1 wherein the proximal end or the distal end of the airway device comprises a circumferential flange and wherein width of the opening in the sheet is dimensioned to be smaller than the width/diameter of the circumferential flange to prevent the proximal end or the distal end from passing through the opening of the flexible sheet and becoming uncoupled from the airway device.

4. Medical apparatus in accordance with claim 3 wherein the opening of the sheet is dimensioned such that at least a portion of an underside surface of the circumferential flange contacts a portion of the flexible sheet defining the opening to form a seal.

5. A medical apparatus in accordance with claim 1 wherein the flexible sheet extends between two lateral sides with top and bottom portions of the sheet extending therebetween and wherein the top portion comprises a cut-out section for receiving a nose of the user, the cut-out section being flanked by a top left portion and a top right portion of the flexible sheet.

6. A medical apparatus in accordance with claim 5 wherein the opening of the sheet is located below the cut-out section for being positioned substantially above the mouth of the user.

7. A medical apparatus in accordance with claim 1 further comprising a locking member configured to be received and engaged with a locking portion located on an outer wall portion of the passage extending between the proximal end and distal end of the airway device for detachably coupling said medical apparatus with the airway device.

8. A medical apparatus in accordance with claim 7 wherein the locking member comprises a profiled projection member configured for engagement with an opening in the locking portion.

9. Medical apparatus in accordance with claim 7 wherein during use the projection member extends in a transverse direction relative to a longitudinal axis of the passage.

10. Medical apparatus in accordance with claim 7 wherein the projection member extends in an in-use downwardly direction towards a radially central portion of the passage.

11. Medical apparatus in accordance with claim 1 wherein the opening is located at an end portion of the passage engaging wall.

12. (canceled)

13. Medical apparatus in accordance with claim 11 wherein said at least portion of the flexible sheet is integrally formed with the passage engaging wall.

14. (canceled)

15. A mask for use in combination with an endoscopy bite block including a front piece to fit over a patient's mouth which has a central opening communicating with a passage extending into the patient's mouth to receive and pass an endoscopic tube, the mask comprising:
a flexible sheet adapted to be placed over a user's face; wherein the flexible sheet comprises opening to allow the passage of the bite block to be passed therethrough, the opening being dimensioned to prevent the front piece from being passed through the opening, and wherein the flexible sheet is substantially impervious to air or liquid, and
a passage engaging wall formed integrally with said flexible sheet, the passage engaging wall being dimensioned for surrounding and engaging an outer wall of the passage of the mouthpiece.

16. (canceled)

17. A mask in accordance with claim 15 wherein the passage extends between a proximal end located adjacent the central opening on the front piece and a distal end adapted to be inserted into the mouth of the patient and being located away from the front piece, wherein the distal end comprises a circumferential flange and wherein width of the opening of the mask is dimensioned to be smaller than the width or diameter of the circumferential flange to prevent the distal end of the passage from passing through the opening of the mask and becoming uncoupled from the endoscopy bite block.

18. A mask in accordance with claim 15 wherein the flexible sheet extends between two lateral sides with top and bottom portions of the sheet extending therebetween and wherein the top portion comprises a cut-out section for receiving a nose of the user, the cut-out section being flanked by a top left portion and a top right portion of the flexible sheet.

19. (canceled)

20. A mask in accordance with claim 15 further comprising a locking member configured to be received and engaged with a locking portion located on an outer wall portion of the passage for detachably coupling said mask with the bite block.

21. A mask in accordance with claim 20 wherein the locking member comprises a profiled and flexible projection member configured for engagement with an opening in the locking portion.

22. A mask in accordance with claim 21 wherein, during use, the projection member extends in a transverse direction relative to a longitudinal axis of the passage.

23. A mask in accordance with claim 21 wherein the projection member extends in an in-use downwardly direction towards a radially central portion of the passage.

24. A mask in accordance with claim 15 wherein the opening is located at an end portion of the passage engaging wall and wherein a portion of the flexible sheet, during use, engages with a back wall of the front piece for the bite block.

25-28. (canceled)

29. An endoscopy bite block in combination with a mask, wherein the bite block comprises a front piece for fitting over a patient's mouth, the front piece having a central opening communicating with a passage extending into the patient's mouth to receive and pass an endoscopic tube therethrough and wherein the mask comprises:
a flexible sheet adapted to be placed over a user's face; wherein the flexible sheet comprises opening to allow the passage to be passed therethrough, the opening being dimensioned to prevent the front piece from being passed through the opening and wherein the flexible sheet is substantially impervious to air or liquid, and
a passage engaging wall formed integrally with said flexible sheet, the passage engaging wall being dimensioned for surrounding and engaging an outer wall of the passage of the mouthpiece.

30-42. (canceled)
